SEQUENCE LISTING (20 sequences)

SEQ ID NO: 1 - DNA, 186 bp, Homo sapiens
tatgttgctaccagagacaactgctgcatcttagatgaaagattcggtagttattgtccaactacctgtggcattgcagattccctgtctacttatcaaaccaaagtagacaaggatctacagtctttggaagacatcttacatcaagttgaaaacaaaacatcagaagtcaaacagctgataaaa

SEQ ID NO: 2 - DNA, 72 bp, Homo sapiens
gcagatagtggtgaaggtgactttctagctgaaggaggaggcgtgcgtggcccaagggttgtggaaagacat

SEQ ID NO: 3 - protein, 24 aa, Homo sapiens
ADSGEGDFLAEGGGVRGPRVVERH

SEQ ID NO: 4 - protein, 24 aa, Homo sapiens
  MISC_FEATURE (23)..(23): any amino acid residue
ADSGEGDFLAEGGGVRGPRVVEXH

SEQ ID NO: 5 - protein, 24 aa, Homo sapiens
  MISC_FEATURE (19)..(19): any amino acid residue
ADSGEGDFLAEGGGVRGPXVVERH

SEQ ID NO: 6 - protein, 24 aa, Homo sapiens
  MISC_FEATURE (17)..(17): any amino acid residue
ADSGEGDFLAEGGGVRXPRVVERH

SEQ ID NO: 7 - protein, 24 aa, Homo sapiens
  MISC_FEATURE (16)..(16): any amino acid residue
ADSGEGDFLAEGGGVXGPRVVERH

SEQ ID NO: 8 - protein, 24 aa, Homo sapiens
  MISC_FEATURE (7)..(7): any amino acid residue
ADSGEGXFLAEGGGVRGPRVVERH

SEQ ID NO: 9 - protein, 24 aa, Homo sapiens
  MISC_FEATURE (6)..(6): any amino acid residue
ADSGEXDFLAEGGGVRGPRVVERH

SEQ ID NO: 10 - protein, 24 aa, Homo sapiens
  MISC_FEATURE (4)..(4): any amino acid residue
ADSXEGDFLAEGGGVRGPRVVERH

SEQ ID NO: 11 - protein, 24 aa, Homo sapiens
  MISC_FEATURE (4)..(4): any amino acid residue
ADSXEXDFLAEGGGVRXPRVVERH

SEQ ID NO: 12 - protein, 24 aa, Homo sapiens
  MISC_FEATURE (7)..(7): any amino acid residue
ADSGEGXFLAEGGGVRGPXVVERH

SEQ ID NO: 13 - protein, 24 aa, Homo sapiens
ADSGEGAFLAEGGGVRGPAVVERH

SEQ ID NO: 14 - protein, 24 aa, Homo sapiens
ADSGEGDFLAEGGGVAGPRVVERH

SEQ ID NO: 15 - protein, 78 aa, Homo sapiens
ADSGEGDFLAEGGGVRGPRVVERHQSACKDSDWPFCSDEDWNYKCPSGCRMKGLIDEVNQDFTNRINKLKNSLFEYQK

SEQ ID NO: 16 - DNA, 234 bp, Homo sapiens
gcagatagtggtgaaggtgactttctagctgaaggaggaggcgtgcgtggcccaagggttgtggaaagacatcaatctgcctgcaaagattcagactggcccttctgctctgatgaagactggaactacaaatgcccttctggctgcaggatgaaagggttgattgatgaagtcaatcaagattttacaaacagaataaataagctcaaaaattcactatttgaatatcagaag

SEQ ID NO: 17 - protein, 80 aa, Homo sapiens
ARPAKAAATQKKVERKAPDAGGCLHADPDLGVLCPTGCQLQEALLQQERPIRNSVDELNNNVEAVSQTSSSSFQYMYLLK

SEQ ID NO: 18 - DNA, 240 bp, Homo sapiens
gctcgtccagccaaagcagctgccactcaaaagaaactagaaagaaaagcccctgatgctggaggctgtcttcacgctgacccagacctgggggtgttgtgtcctacaggatgtcagttgcaagaggctttgctacaacaggaaaggccaatcagaaatagtgttgatgagttaaataacaatgtggaagctgtttcccagacctcctcttcttcctttcagtacatgtatttgctgaaa

SEQ ID NO: 19 - protein, 62 aa, Homo sapiens
YVATRDNCCILDERFGSYCPTTCGIADFLSTYQTKVDKDLQSLEDILHQVENKTSEVKQLIK

SEQ ID NO: 20 - DNA, 186 bp, Homo sapiens; identical to SEQ ID NO: 1

(19) United States
(12) Patent Application Publication
(10) Pub. No.: US 2004/0039157 A1
(43) Pub. Date: Feb. 26, 2004

(54) Anti-angiogenic peptides

(76) Inventors: Carolyn Staton, Sheffield (GB); Claire Lewis, Sheffield (GB); Jeffery Robinson, Surrey (GB)

Correspondence Address: THOMAS HOXIE, NOVARTIS, CORPORATE INTELLECTUAL PROPERTY, ONE HEALTH PLAZA 430/2, EAST HANOVER, NJ, 07936-1080, US

(21) Appl. No.: 10/363,369

(22) PCT Filed: Sep. 3, 2001
(86) PCT No.: PCT/GB01/03926

(30) Foreign Application Priority Data
Sep. 1, 2000 (GB) 0021475.9
Nov. 9, 2000 (GB) 0027395.3
Jul. 23, 2001 (GB) 0117737.7

(51) Int. Cl. C07K 7/08

(52) U.S. Cl. 530/324

(57) ABSTRACT

The invention relates to anti-angiogenic peptides derived from fibrinogen; pharmaceutical compositions comprising said peptides; nucleic acids encoding said peptides and methods to treat animals, preferably humans, suffering from diseases which would benefit from the inhibition of angiogenesis.

The invention relates to the anti-angiogenic effects of peptides derived from fibrinogen.

Angiogenesis, the development of new blood vessels from an existing vascular bed, is a complex multistep process that involves the degradation of components of the extracellular matrix and then the migration, proliferation and differentiation of endothelial cells to form tubules and eventually new vessels. Angiogenesis is important in normal physiological processes including, by example and not by way of limitation, embryo implantation; embryogenesis and development; and wound healing. Excessive angiogenesis is also involved in pathological conditions such as tumour cell growth and non-cancerous conditions such as neovascular glaucoma, rheumatoid arthritis, psoriasis and diabetic retinopathy.

The vascular endothelium is normally quiescent. However, upon activation endothelial cells proliferate and migrate to form microtubules which will ultimately form a capillary bed to supply blood to developing tissues and, of course, a growing tumour. A number of growth factors have been identified which promote/activate endothelial cells to undergo angiogenesis. These include, by example and not by way of limitation; vascular endothelial growth factor (VEGF); transforming growth factor (TGFb); acidic and basic fibroblast growth factor (aFGF and bFGF); and platelet derived growth factor (PDGF) (1,2).

VEGF is a an endothelial cell-specific growth factor which has a very specific site of action, namely the promotion of endothelial cell proliferation, migration and differentiation. VEGF is a dimeric complex comprising two identical 23 kD polypeptides. The monomeric form of VEGF can exist as four distinct polypeptides of different molecular weight, each being derived from an alternatively spliced mRNA. Of the four monomeric forms, two exist as membrane bound VEGF and two are soluble. VEGF is expressed by a wide variety of cell/tissue types including embryonal tissues; proliferating keratinocytes; macrophages; tumour cells. Studies (2) have shown VEGF is highly expressed in many tumour cell-lines including glioma and AIDS-associated Kaposi's sarcoma. VEGF activity is mediated through VEGF specific receptors expressed by endothelial cells and tumour cells. Indeed, VEGF receptors are up-regulated in endothelial cells which infiltrate tumours thereby promoting tumour cell growth.

bFGF is a growth factor which functions to stimulate the proliferation of fibroblasts and endothelial cells. bFGF is a single polypeptide chain with a molecular weight of 16.5 Kd. Several molecular forms of bFGF have been discovered which differ in the length at their amino terminal region. However the biological function of the various molecular forms appears to be the same. bFGF is produced by the pituitary gland and is encoded by a single gene located on human chromosome 4.

A number of endogenous inhibitors of angiogenesis have been discovered, examples of which are angiostatin and endostatin, which are formed by the proteolytic cleavage of plasminogen and collagen XVIII respectively. Both of these factors have been shown to suppress the activity of pro-angiogenic growth factors such as vascular VEGF and bFGF. Both also suppress endothelial cell responses to VEGF and bFGF in vitro, and reduce the vascularisation and growth of experimental tumours in animal models.

Fibrinogen, the soluble circulating precursor of fibrin, is a dimeric molecule containing pairs of non-identical chains, (ie the α-,β- and γ-chains). These are arranged as three discrete domains, the two outer D-domains and the central E-domain (4). Fibrinogen can be digested either by plasmin or thrombin.

The first step in plasmin cleavage of fibrinogen is the cleavage of the α chain C-terminal domain. Plasmin then cleaves the two D domains from the one E domain (consisting of the NH2 terminal regions of the α-,β- and γ-chains held together by disulphide bonds) and numerous smaller fragments including a small peptide, beta1-42 (amino terminal of the β-chain) (5). Thrombin, on the other hand, produces a fibrin monomer and two copies of fibrinopeptides A and B (4). Fibrinogen has been shown to accumulate around leaky blood vessels in solid tumours(5), Fibrinogen has also been shown to polymerise at host-tumour interface to form fibrin networks that promote tumour angiogenesis by supporting the adhesion, migration, proliferation and differentiation of endothelial cells (7).

The fibrin E-fragment (FnE-fragment), produced by the proteolytic cleavage of fibrin, stimulates angiogenesis in the chorioallantoic membrane assay (8). Furthermore, the amount of this protein present in invasive breast carcinomas positively correlates with the degree of tumour vascularity (5).

A potent, new inhibitor of angiogenesis, which is a 50 kDa proteolytic fragment of fibrinogen, fibrinogen E, is disclosed in our co-pending application, PCT/GB01/02079, which is incorporated by reference. We have now identified a domain within the fibrinogen E fragment which has the same anti-angiogenic activity as the very much larger fibrinogen E fragment. The domain is located at the amino terminus of the α chain and is referred to as α1-24. A peptide derived from the domain has anti-angiogenic activity. We have also identified modified α1-24 peptides which retain the anti-angiogenic activity of the unmodified α1-24 peptide.

According to a first aspect of the invention there is provided a polypeptide comprising an amino acid sequence selected from the group consisting of:

i) a peptide of the sequence:

A D S X E X X F L A E G G G V X X P X V V E X H

wherein X is any amino acid residue;

ii) a peptide as represented in (i) wherein amino acid residue X is selected from the following group: alanine, valine, leucine, isoleucine, proline; and

iii) a peptide represented in (i) or (ii) which has anti-angiogenic activity.

Reference to anti-angiogenic activity is determined by assays hereindisclosed. For example, the polypeptides of the invention are tested by in vitro assays which include the inhibition of endothelial cell mediated tubule formation, inhibition of endothelial cell migration, inhibition of VEGF and bFGF induced endothelial cell proliferation and endothelial cell cytotoxicity assays. Polypeptides can also be tested in vivo using murine tumour models as hereindisclosed.

In a preferred embodiment of the invention said polypeptide comprises an amino acid sequence selected from the following group:

A D S G E G D F L A E G G G V R G P R V V E R H
A D S G E G D F L A E G G G V R G P R V V E X H
A D S G E G D F L A E G G G V R G P X V V E R H
A D S G E G D F L A E G G G V R X P R V V E R H
A D S G E G D F L A E G G G V X G P R V V E R H
A D S G E G X F L A E G G G V R G P R V V E R H
A D S G E X D F L A E G G G V R G P R V V E R H
A D S X E G D F L A E G G G V R G P R V V E R H
A D S X E X D F L A E G G G V R X P R V V E R H
A D S G E G X F L A E G G G V R G P X V V E R H

In a further preferred embodiment of the invention said peptide comprises an amino acid sequence wherein X is alanine.

In a yet further preferred embodiment of the invention said polypeptide comprises the amino acid sequence:

A D S G E G D F L A E G G G V R G P R V V E R H

Reference to α1-24 peptide is reference to a peptide which has the sequence:

A D S G E G D F L A E G G G V R G P R V V E R H,

or active peptides derived from this sequence which have been modified by, addition, deletion, or substitution of at least one amino acid residue.

In a preferred embodiment of the invention said polypeptide is at least 24 amino acid residues in length. Preferably said peptide consists of the amino acid sequence A D S G E G D F L A E G G G V R G P R V V E R H, or fragment thereof.

Reference to fragment is reference to a peptide derived from the α1-24 peptide which retains anti-angiogenic activity. Such fragments may be 3 amino acids in length; preferably said fragments are 4, 5, 6, 7, 8, 9, 10, 11, 12, 13, 14, 15, 16, 17, 18, 19, 20, 21, 22 or 23 amino acid residues in length.

It will be apparent to one skilled in the art that modification to the amino acid sequence of polypeptides comprising α1-24 could enhance the binding and/or stability of the polypeptide with respect to its target sequence. In addition, modification of the polypeptide may also increase the in vivo stability of the polypeptide thereby reducing the effective amount of polypeptide necessary to inhibit angiogenesis. This would advantageously reduce undesirable side effects which may result in vivo. Modifications include, by example and not by way of limitation, acetylation and amidation.

In a preferred embodiment of the invention said polypeptide comprising the α1-24 sequence is acetylated. Preferably said acetylation is to the amino terminus of said polypeptide. More preferably still the amino terminal alanine amino acid of the α1-24 peptide is acetylated.

In a further preferred embodiment of the invention said polypeptide comprising the α1-24 sequence is amidated. Preferably said amidation is to the carboxyl-terminus of said polypeptide. More preferably still the carboxy-terminal histidine amino acid is amidated.

In a further preferred embodiment of the invention the α1-24 peptide, or fragment thereof, is modified by both acetylation and amidation. Preferably said acetylation is to the amino-terminal alanine amino acid of the α1-24 peptide and said amidation is to the carboxyl-terminal histidine of the α1-24 peptide.

It will be apparent to one skilled in the art that fragments of the α1-24 peptide as herein disclosed, are susceptible to modifications such as acetylation and/or amidation.

Alternatively or preferably, said modification includes the use of modified amino acids in the production of recombinant or synthetic forms of polypeptides comprising α1-24 peptide.

It will be apparent to one skilled in the art that modified amino acids include, by way of example and not by way of limitation, 4-hydroxyproline, 5-hydroxylysine, N⁶-acetyllysine, N⁶-methyllysine, N⁶,N⁶-dimethyllysine, N⁶,N⁶,N⁶-trimethyllysine, cyclohexyalanine, D-amino acids, ornithine. Other modifications include amino acids with a C₂, C₃ or C₄ alkyl R group optionally substituted by 1, 2 or 3 substituents selected from halo ( eg F, Br, I), hydroxy or C₁—C₄ alkoxy.

In a further preferred embodiment of the invention there is provided a polypeptide according to the invention which polypeptide comprises at least one modified amino acid wherein X denotes the position of said modified amino acid.

The incorporation of modified amino acids may confer advantageous properties on polypeptides comprising α1-24. For example, the incorporation of modified amino acids may increase the affinity of the polypeptide for its binding site, or the modified amino acids may confer increased in vivo stability on the polypeptide thus allowing a decrease in the effective amount of therapeutic polypeptide administered to a patient.

It will also be apparent to one skilled in the art that fragments of α1-24 that retain anti-angiogenic activity could be recovered by fractionation of the intact polypeptide using, for example, proteolytic enzymes. Alternatively, fragments could be synthesised de novo and also modified by, for example, cyclisation. Cyclisation is known in the art, (see Scott et al Chem Biol (2001), 8:801-815; Gellerman et al J. Peptide Res (2001), 57: 277-291; Dutta et al J. Peptide Res (2000), 8: 398-412; Ngoka and Gross J Amer Soc Mass Spec (1999), 10:360-363.

In a preferred embodiment of the invention the polypeptides according to the invention are modified by cyclisation.

According to a further aspect of the invention there is provided a nucleic acid molecule comprising DNA sequences selected from:

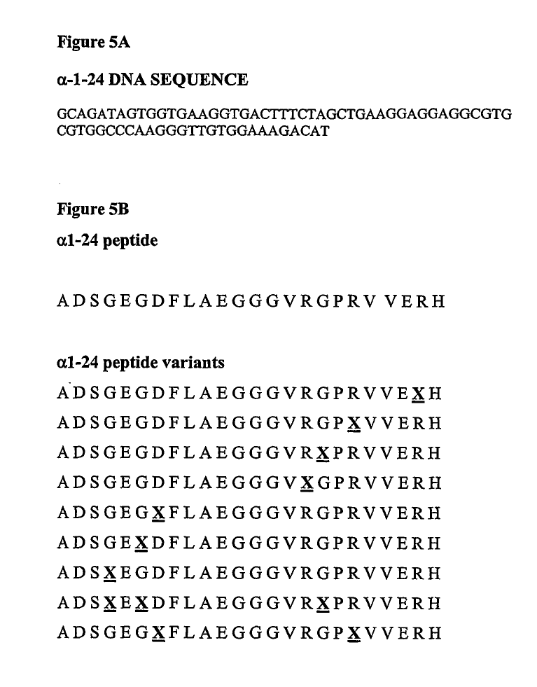

i) the DNA sequence as represented in FIG. 5a;

ii) the DNA sequence as represented in FIG. 5a which has been modified by addition, deletion, or substitution of at least one nucleotide base within at least one codon to encode a modified peptide according to the invention;

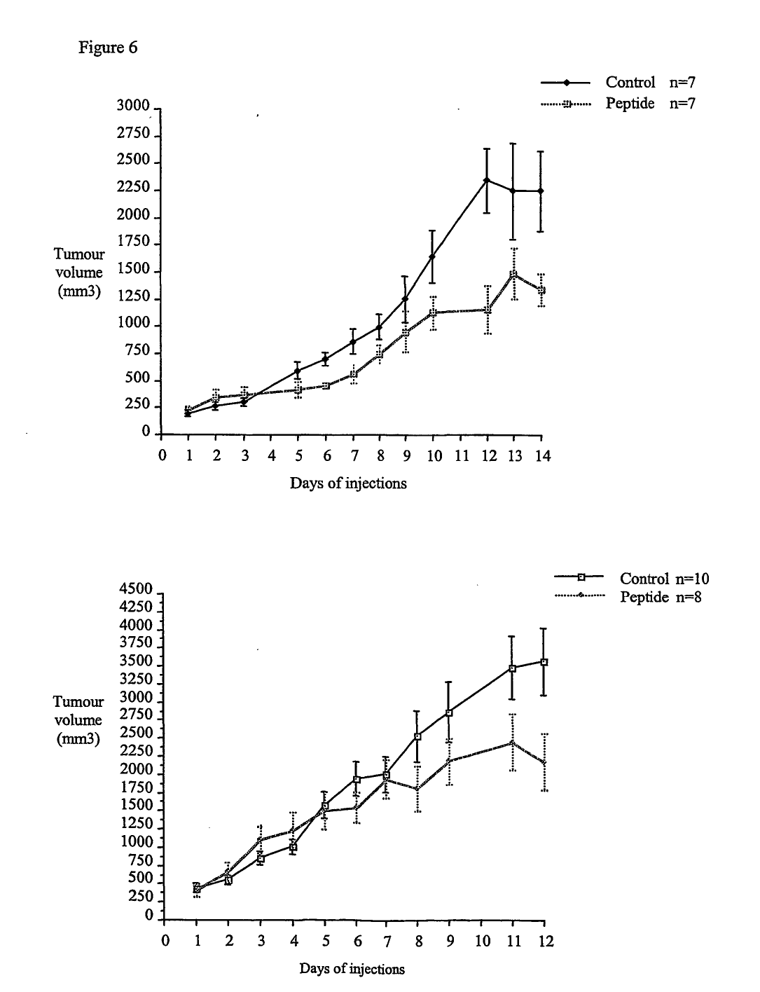

iii) DNA sequences which hybridise to the sequences presented in FIG. 6 which encode a peptide having anti-angiogenic activity; and

iv) DNA sequences which are degenerate as a result of the genetic code to the DNA sequences defined in (i), (ii) or (iii).

In a preferred embodiment of the invention there is provided an isolated nucleic acid molecule which anneals under stringent hybridisation conditions to the sequences described in (i), (ii), (iii) and (iv) above.

Stringent hybridisation/washing conditions are well known in the art. For example, nucleic acid hybrids that are stable after washing in 0.1×SSC,0.1% SDS at 60° C. It is well known in the art that optimal hybridisation conditions can be calculated if the sequence of the nucleic acid is known. Typically, hybridisation conditions uses 4-6×SSPE (20×SSPE contains 175.3 g NaCl, 88.2 g NaH₂PO₄ H₂O and 7.4 g EDTA dissolved to 1 litre and the pH adjusted to 7.4); 5-10×Denhardts solution (50×Denhardts solution contains 5 g Ficoll (Type 400, Pharmacia), 5 g polyvinylpyrrolidone abd 5 g bovine serum albumen; 100 μg-1.0 mg/ml sonicated salmon/herring DNA; 0.1-1.0% sodium dodecyl sulphate; optionally 40-60% deionised formamide. Hybridisation temperature will vary depending on the GC content of the nucleic acid target sequence but will typically be between 42°-65° C.

According to a further aspect the invention there is provided a pharmaceutical composition comprising a α1-24 peptide, or part thereof,

When administered, the pharmaceutical compositions of the present invention are administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines and optionally other therapeutic agents, such as chemotherapeutic agents.

The pharmaceutical compositions of the invention can be administered by any conventional route, including injection or by gradual infusion over time. The administration may, for example, be oral, intravenous, intraperitoneal, intramuscular, intracavity, subcutaneous, or transdermal.

The compositions of the invention are administered in effective amounts. An “effective amount” is that amount of a composition that alone, or together with further doses, produces the desired response. In the case of treating a particular disease, such as cancer, the desired response is inhibiting the progression of the disease. This may involve slowing the progression of the disease temporarily, although more preferably, it involves halting the progression of the disease permanently. This can be monitored by routine methods.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation.

The pharmaceutical compositions used in the foregoing methods of treatment preferably are sterile and contain an effective amount of α1-24 peptide or nucleic acid encoding α1-24 peptide for producing the desired response in a unit of weight or volume suitable for administration to a patient.

The doses of α1-24 peptide or nucleic acid encoding α1-24 peptide administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

When administered, the therapeutic preparations of the invention are applied in therapeutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention.

α1-24 peptide compositions may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzallconium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as a syrup, elixir or an emulsion.

Compositions suitable for parenteral administration conveniently comprise a sterile aqueous or non-aqueous preparation of α1-24 peptides or nucleic acids, which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono-or di-glycerides. In addition, fatty acids such as oleic acid may be used in the preparation of injectables.

Carrier formulation suitable for oral, subcutaneous, intravenous, intramuscular, etc. administrations can be found in Remington's Pharmaceutical Sciences, Mack Publishing Co., Easton, Pa.

In a preferred embodiment of the invention said pharmaceutical composition modulates angiogenesis. Preferably said modulation is the inhibition of angiogenesis. Preferably said inhibition relates to endothelial cell stimulated angiogenesis.

Alternatively, or preferably, said inhibition is the inhibition of macrophage and/or tumour cell stimulated angiogenesis.

In a further preferred embodiment of the invention said inhibition is mediated by the inhibition of pro-angiogenic factors. Ideally these are either intracellular or cell surface receptors.

More preferably still, said inhibition is mediated via inhibition of the activity of pro-angiogenic growth factors. Ideally said growth factors are selected from: VEGF, bFGF; aFGF; TGFβ; PDGF.

According to a yet further aspect of the invention there is provided the use of a polypeptide comprising α1-24 peptides, or part thereof, in the manufacture of a medicament for use in the treatment of cancer.

Polypeptides which comprise α1-24 peptides can be manufactured by in vitro peptide synthesis using standard peptide synthesis techniques. Alternatively, or preferably, polypeptides can be manufactured by recombinant techniques which are well known in the art.

According to a further aspect of the invention there is provided a vector, wherein said vector includes a nucleic acid molecule which encodes for polypeptides which comprise α1-24 peptides.

Alternatively, vector(s) which include nucleic acid encoding polypeptides which comprise α1-24 peptides can be adapted for recombinant expression.

In a preferred embodiment of the invention said vector is an expression vector adapted for prokaryotic or eukaryotic cell expression. Preferably said eukaryotic vector is adapted for gene therapy.

Typically said adaptation includes, by example and not by way of limitation, the provision of transcription control sequences (promoter sequences) which mediate cell/tissue specific expression. These promoter sequences may be cell/tissue specific, inducible or constitutive.

Promoter is an art recognised term and, for the sake of clarity, includes the following features which are provided by example only, and not by way of limitation. Enhancer elements are cis acting nucleic acid sequences often found 5′ to the transcription initiation site of a gene (enhancers can also be found 3′ to a gene sequence or even located in intronic sequences and is therefore position independent). Enhancers function to increase the rate of transcription of the gene to which the enhancer is linked. Enhancer activity is responsive to trans acting transcription factors (polypeptides) which have been shown to bind specifically to enhancer elements. The binding/activity of transcription factors (please see Eukaryotic Transcription Factors, by David S Latchman, Academic Press Ltd, San Diego) is responsive to a number of environmental cues which include, by example and not by way of limitation, intermediary metabolites or environmental effectors.

Promoter elements also include so called TATA box and RNA polymerase initiation selection (RIS) sequences which function to select a site of transcription initiation. These sequences also bind polypeptides which function, inter alia, to facilitate transcription initiation selection by RNA polymerase.

Adaptations also include the provision of selectable markers and autonomous replication sequences which both facilitate the maintenance of said vector in either the eukaryotic cell or prokaryotic host. Vectors which are maintained autonomously are referred to as episomal vectors.

Adaptations which facilitate the expression of vector encoded genes include the provision of transcription termination/polyadenylation sequences. This also includes the provision of internal ribosome entry sites (IRES) which function to maximise expression of vector encoded genes arranged in bicistronic or multi-cistronic expression cassettes.

These adaptations are well known in the art. There is a significant amount of published literature with respect to expression vector construction and recombinant DNA techniques in general. Please see, Sambrook et al (1989) Molecular Cloning: A Laboratory Manual, Cold Spring Harbour Laboratory, Cold Spring Harbour, N.Y. and references therein; Marston, F (1987) DNA Cloning Techniques: A Practical Approach Vol III IRL Press, Oxford UK; DNA Cloning: F M Ausubel et al, Current Protocols in Molecular Biology, John Wiley & Sons, Inc.(1994).

In a yet further preferred embodiment of the invention there is provided a gene therapy vector comprising the nucleic acid according to the invention.

It will be apparent to one skilled in the art that the delivery of gene therapy vectors either to endothelial cells or tumour cells target the production of polypeptides comprising α1-24 peptides to the vicinity of the tumour thereby augmenting the anti-angiogenic effect of polypeptides comprising α1-24.

According to a yet further aspect of the invention there is provided a cell transformed/transfected with the nucleic acid according to the invention. Ideally said nucleic acid is the vector according to the invention.

According to a further aspect of the invention there is provided a method for the production of polypeptides comprising α1-24 including:

i) providing a cell according to the invention;

ii) providing conditions conducive to the manufacture of polypeptides comprising α1-24; and

iii) purifying said polypeptides from a cell, or a cells culture environment.

According to yet still a further aspect of the invention there is provided a non-human, transgenic animal characterised in that said animal incorporates a nucleic acid molecule encoding a polypeptide comprising α1-24 into its genome.

It will be apparent to one skilled in the art that the provision of non-human transgenic animals genetically modified by the provision of a transgene(s) encoding polypeptides which comprise α1-24 is an alternative source of active polypeptide. It is well known in the art that transgenic animals can be used to make various therapeutic polypeptides.

In a preferred embodiment of the invention said transgene is of human origin.

In a further aspect of the invention there is provided a method to treat an animal which would benefit from inhibition of angiogenesis comprising:

i) administering an effective amount of an agent comprising α1-24 to an animal to be treated;

ii). monitoring the effects of said agent on the inhibition of angiogenesis.

In a preferred method of the invention said treatment is the inhibition of tumour development.

In an alternative method of treatment, polypeptides comprising α1-24 are additionally conjugated, associated or crosslinked to an agent which augments the anti-angiogenic effect of the polypeptide.

Typically the agent could be a cytotoxic agent, another anti-angiogenic agent, a prodrug activating enzyme, a chemotherapeutic agent, a pro-coagulant agent or immunomodulatory factor.

Examples of these are well known in the art, for example, and not by way of limitation cytotoxins, such as ricin A-chain or diphtheria toxin; antagonists of the key pro-angiogenic factors in tumours (eg VEGF, bFGF, TNF alpha, PDGF) would include neutralising antibodies or receptors for these factors, or tyrosine kinase inhibitors for their receptors (eg. SU5416 for the VEGF receptor, Flk-1/KDR); prodrug activating enzymes such as, human simplex virus-thymidine kinase HSV-TK, which activates the prodrug, ganciclovir when it is then admininistered systemically; chemotherapeutic agents, such as neocarzinostatin; cisplatin; carboplatin; cyclosphosphamide; melphalan; carmusline; methotrexate; 5-fluorouracil; cytarabine; mercaptopurine; daunorubicin; doxorubicin; epirubicin; vinblastine; vincristine; dactinomycin; mitomycin C; taxol; L-asparaginase; G-CSF; an enediyne such as chalicheamicin or esperamicin; chlorambucil; ARA-C; vindesine; bleomycin; and etoposide.

In addition, or alternatively, the cell surface domain of human tissue factor (this truncated form of tissue factor (tTF) could also be associated with α1-24. Truncated TF has limited anti-endothelial activity when free in the circulation, but becomes an effective and selective thrombogen (ie it causes extensive thrombosis and coagulation in blood vessels) when targeted to the surface of tumor endothelial cells.

An example of an immunomodulatory factor is the Fc effector domain of human IgG1. This binds natural killer (NK) cells and also the C1q protein that initiates the complement cascade. NK cells and complement then activate a powerful cytolytic response against the targeted endothelial cells.

It will be apparent that the above combinations of α1-24 and therapeutic agents will also have benefit with respect to the treatment of other conditions/diseases which are dependent on angiogenesis. For example, neovascular glaucoma, rheumatoid arthritis, psoriasis and diabetic retinopathy.

In a yet further alternative method of treatment, said gene therapy vector includes, and therefore said nucleic acid encoding a polypeptide comprising α1-24 is provided with, nucleic acid encoding an agent which augments the anti-angiogenic effect of α1-24.

According to a yet further aspect of the invention there is provided an imaging agent comprising α1-24.

It will be apparent to the skilled artisan that polypeptides comprising α1-24can be used to target imaging agents to, for example, tumours, to identify developing tumours or to monitor the effects of treatments to inhibit tumour growth. It will also be apparent that the combined therapeutic compositions which comprise both α1-24 and a further anti-angiogenic agent may be further associated with an imaging agent to monitor the distribution of the combined therapeutic composition and/or to monitor the efficacy of said combined composition.

Methods used to detect imaging agents are well known in the art and include, by example and not by way of limitation, positron emission tomographic detection of F¹⁸ and C¹¹ compounds.

An embodiment of the invention will now be described, by example only, and with reference to the following figures:

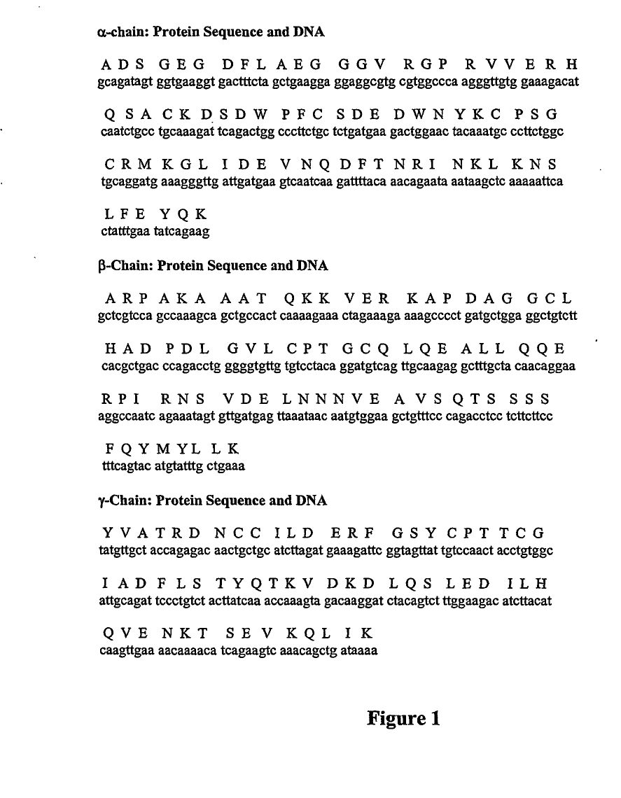

FIG. 1 represents the nucleic acid and amino acid sequences of the α-β-γ-polypeptides of fibrinogen E;

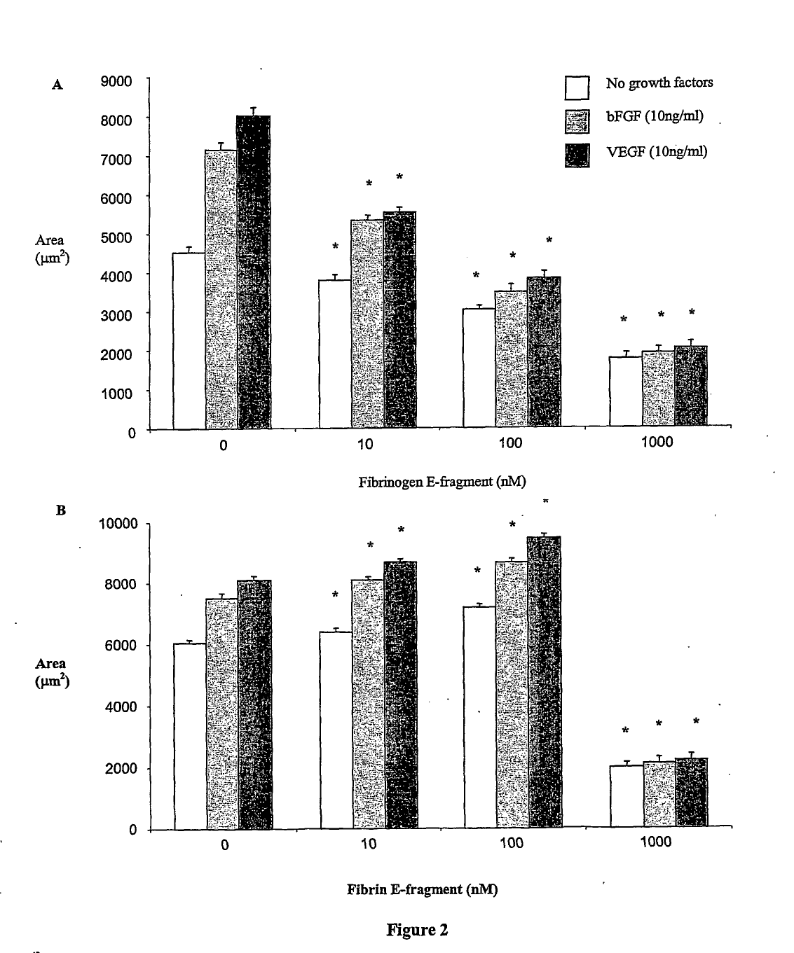

FIG. 2 represents a comparison of the effects of Fibrinogen E-fragment (panel A) and Fibrin E-fragment (panel B) on tubule formation by HuDMECs in vitro. Mean (±SEM) area covered by tubule formation in the absence (empty bars) or presence (coloured bars) of VEGF or bFGF. Each test condition was carried out in three replicate wells, with total area measured in three randomly selected fields of view per well (n=9).*p<0.005 with respect to the relevant control group;

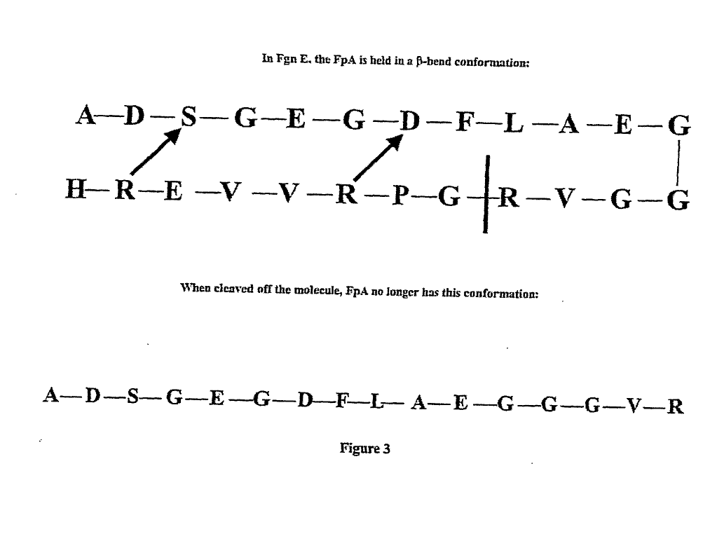

FIG. 3 represents a schematic diagram showing the difference in structure between the anti-angiogenic Fibrinogen E-fragment (A) and the pro-angiogenic Fibrin E-fragment (B). The only difference is the presence (Fibrinogen E-fragment) or absence (Fibrin E-fragment) of the 16 amino acid Fibrinopeptide A;

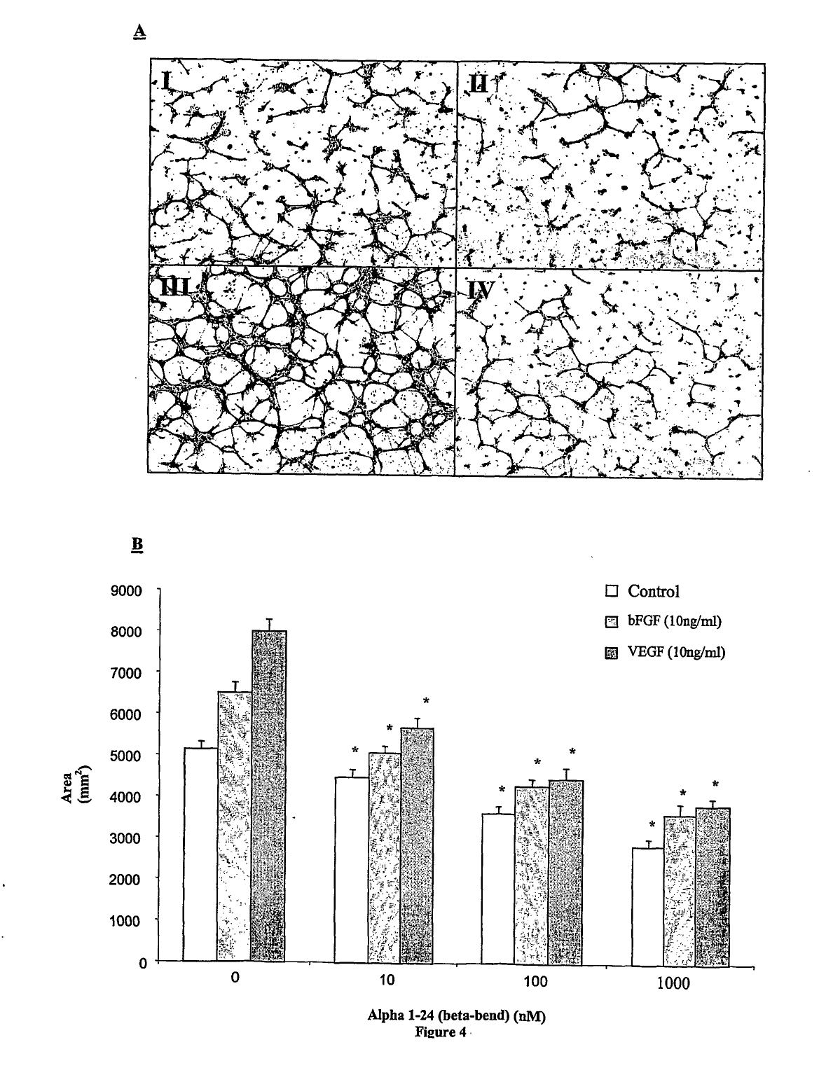

FIG. 4 represents the effects of α1-24 (β-bend) on tubule formation in the absence or presence of VEGF or bFGF by SVEC4-10 cells in vitro. Upper Panel (A): Tubule formation in the GF-reduced Matrigel assay (×40 objective) in the absence of exogenous factors (control) (I), or the presence of 100 nM α1-24 (II), 10 ng/ml VEGF (III) or 10 ng/ml VEGF+100 nM α1-24. Lower panel (B): mean (±SEM) area of tubule formation in the absence of (empty bars) or presence (grey bars) of VEGF or bFGF. n=9, *p<0.03 wit respect to relevant control group;

FIG. 5A is the nucleic acid sequence encoding the α1-24 peptide; FIG. 5B represents the linear amino acid sequence of the α1-24 peptide and the amino acid sequence of various modified α1-24 peptides;

FIG. 6 illustrates the in vivo effect of unmodified α1-24 peptide on tumour growth in mice. The effect of daily injections (i.p.) of 25 ug/kg α1-24 peptide in PBS or PBS alone (control) on growth of CT26 tumours in Balb/c mice, (data from 2 separate experiments);

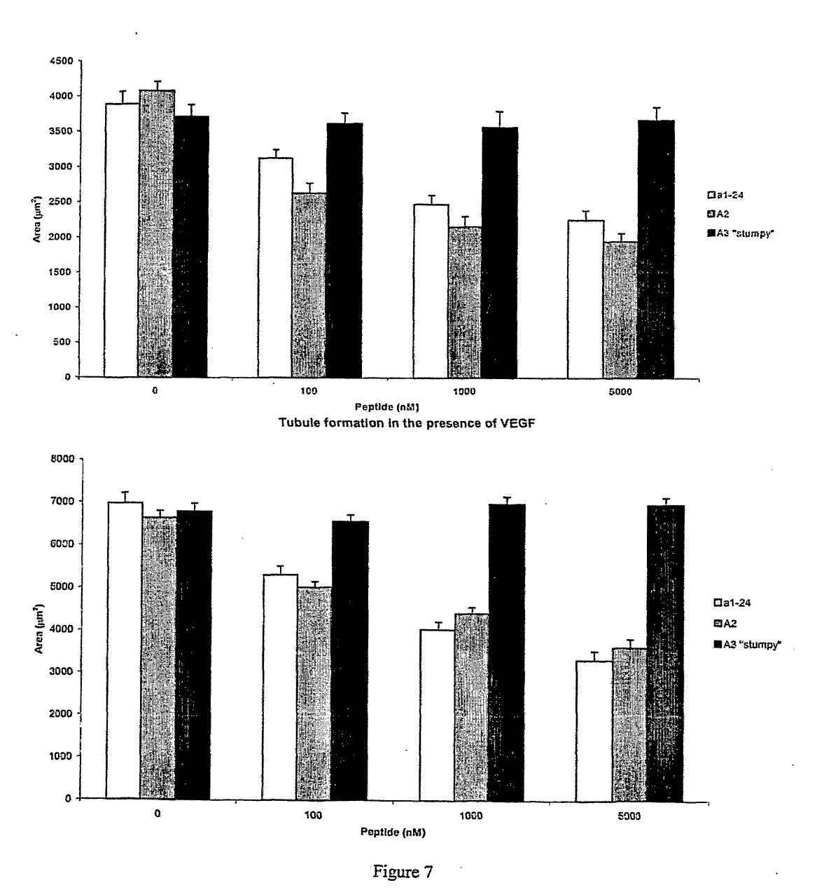

FIG. 7 represents a comparison of the anti-angiogenic activity of an arginine 23 to alanine substitution (R23A); α1-24 control peptide and a truncated α1-24 (amino acids 17-24;

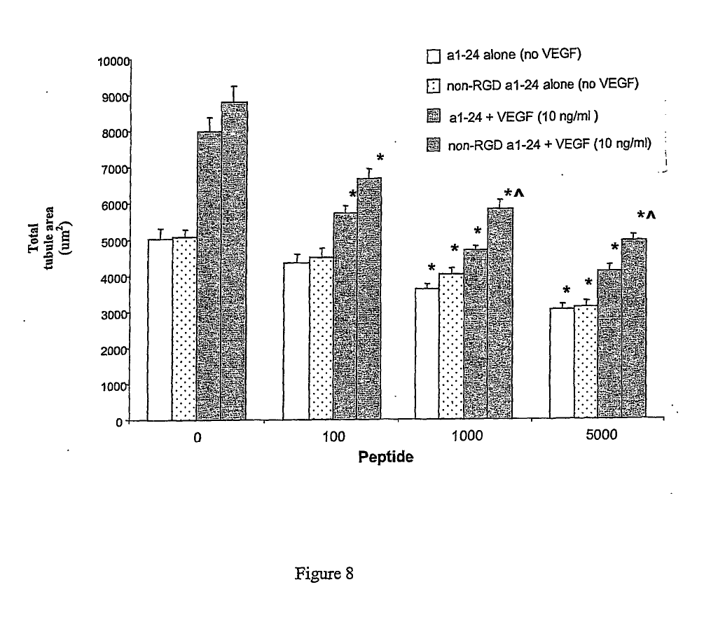

FIG. 8 represents the anti-angiogenic activity of an alanine substituted α1-24 peptide (G4A, G6A,G17A);

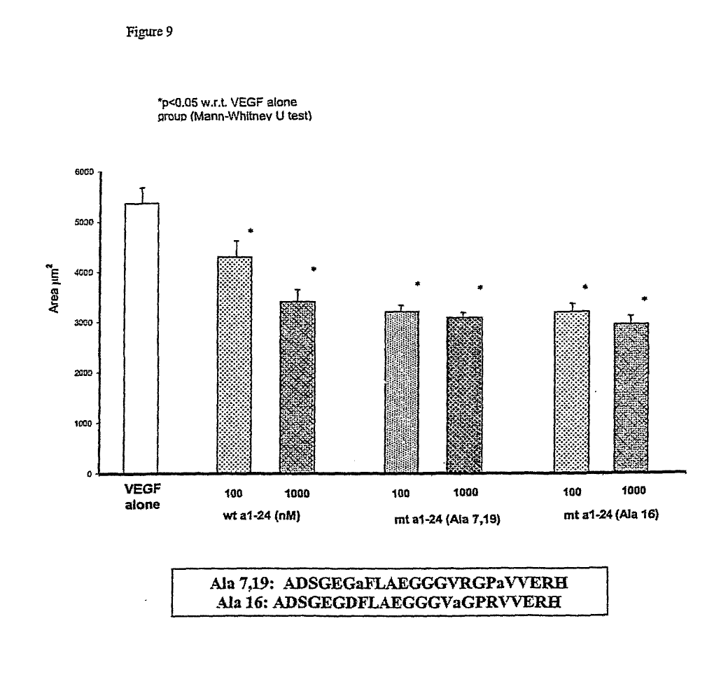

FIG. 9 represents the anti-angiogenic activity of an alanine substituted α1-24 peptide (D7A and R19) and (R16A);

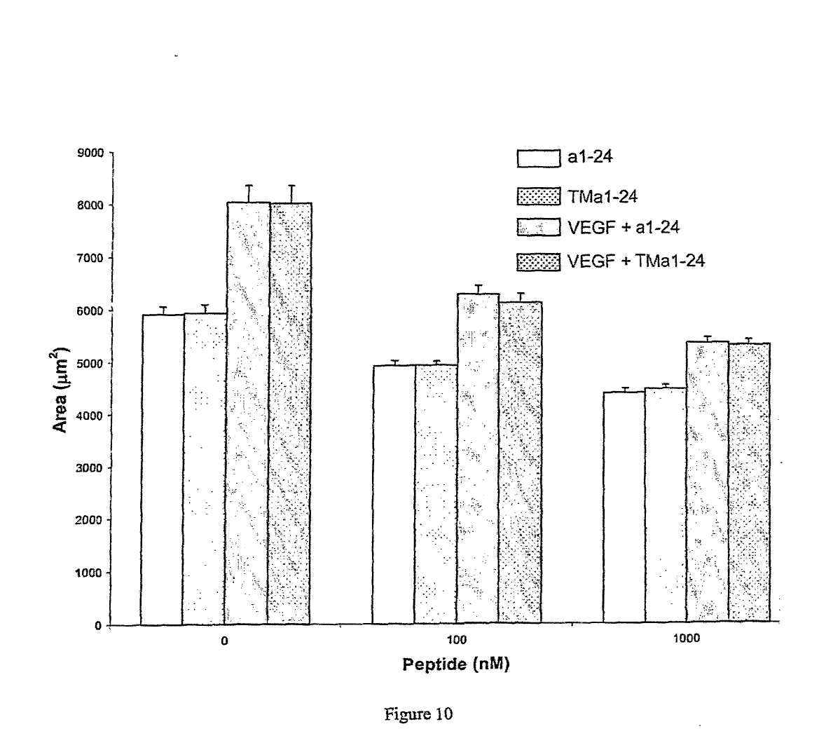

FIG. 10 represents the anti-angiogenic activity of terminal modification (acetylation and amidation) to the α1-24 peptide. The effect of terminal modification (TMa) on the inhibitory effect of alpha1-24 peptide on tubule formation by HuDMECs (with or without 10 ng/ml VEGF) in vitro; and

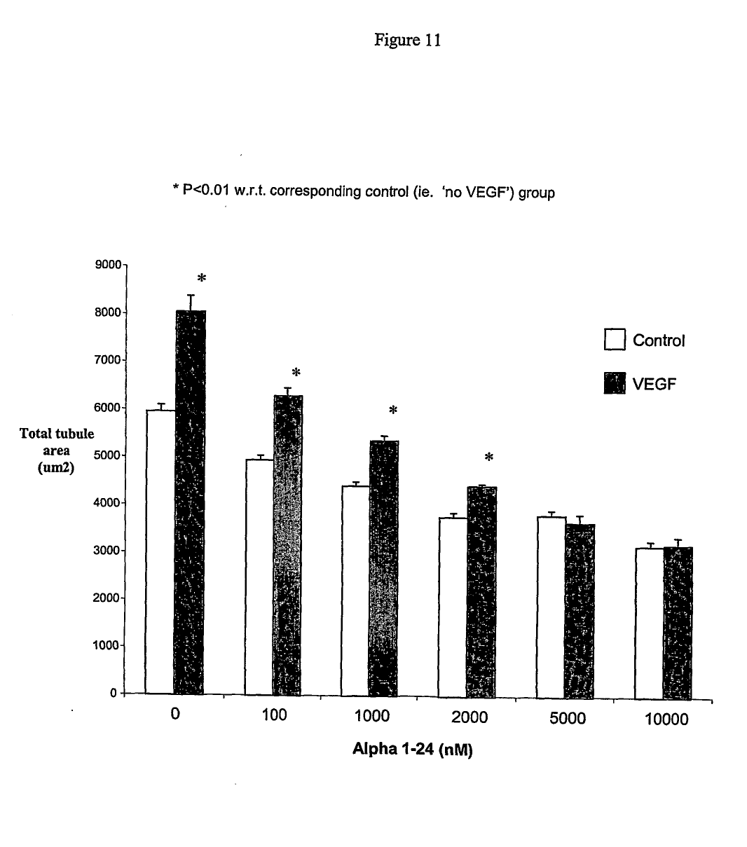

FIG. 11 represents the effect of a wider range of concentrations of α1-24 peptide on tubule formation by HuDMECs (in the presence or absence of 10 ng/ml VEGF) in vitro.

### MATERIALS AND METHODS

Adult human dermal microvascular endothelial cells (HuDMECs) were obtained commercially (TCS Biologicals, Buckinghamshire, United Kingdom) and cultured in microvascular endothelial cell growth medium (EGM). This medium contains heparin (10 ng/ml), hydrocortisone, human epidermal growth factor (10 ng/ml), human fibroblast growth factor (10 ng/ml) (such endothelial growth factors are necessary for routine passaging of HuDMECs in culture) and dibutyryl cyclic AMP. This was supplemented with 5% heat-inactivated FCS, 50 μg/ml gentamicin and 50 ng/ml amphotericin B (TCS Biologicals, United Kingdom). Murine endothelial cells (SVEC 4-10) were obtained from the ATCC and cultured in DMEM+10% FCS. Cells were grown at 37° C. in a 100% humidified incubator with a gas phase of 5% CO₂ and routinely screened for Mycoplasma. Prior to their use in the assays indicated below, HuDMECs were grown to 80% confluency, incubated in DMEM+1% FCS for 2 h, then harvested with 0.05% trypsin solution, washed twice and resuspended to the cell density required for each assay (see below).

Proteins and Peptides.

Human fibrinogen (Fgn) E-fragment was purchased from Diagnostica Stago, Asnieres, France. This was produced by plasmin cleavage of fibrinogen and purified by electrophoresis, immuno-electrophoresis, ion exchange and gel filtration. To generate human Fibrin (Fn) E-fragment, Fgn E-fragment was digested with human thrombin (Sigma-Aldrich Co, Dorset, United Kingdom), as previously described (10). To control for the possible effects of trace amounts of thrombin in the Fn E-fragment preparation on our assays, the same amount of thrombin (0.5 U/ml) was added to control media used in experiments using Fn E-fragment. HPLC-purified FpA was obtained commercially from Bachem Ltd., Saffron Walden, United Kingdom. This peptides was included in the study as this peptide is cleaved from Fgn E-fragment in the production of FnE and will be present in the assays in trace amounts. The beta-bend (α1-24) was generated by standard peptide synthesis methods using FMOC amino acids and a synthesis machine. The purity of the peptide was checked by mass spectroscopy.

Tubule Formation Assay.

24 well plates were coated with 30 μl/well of growth factor-reduced (GF-reduced) Matrigel (Becton Dickinson Labware, Bedford, Mass.). Endothelial cells plated on this matrix migrate and differentiate into tubules within 6 h of plating as described previously (14). HuDMECs or SVEC 4-10 cells were seeded at a density of 4×10⁴ cells/ml and incubated for 6 h in 500 μl of either DMEM+1% FCS alone (control), or this medium±10 ng/ml VEGF or bFGF in the presence or absence of fibrinogen E-fragment, fibrin E-fragment, FpA or α1-24. Assessment of tubule formation involved fixing the cell preparation in 70% ethanol at 4° C. for 15 minutes, rinsing in PBS and staining with haematoxylin and eosin. Three random fields of view in 3 replicate wells for each test condition were visualised under low power (×40 magnification), and colour images captured using a Fuji digital camera linked to a Pentium III computer (containing a frame grabber board). Tubule formation was assessed by counting the number of tubule branches and the total area covered by tubules in each field of view using image analysis software supplied by Scion Image.

Migration Assay

The Boyden chamber technique was adapted from (13) and used to evaluate HuDMEC migration across a porous membrane towards a concentration gradient of either VEGF (10 ng/ml) or bFGF (10 ng/ml). The Neuro Probe 48 well microchemotaxis chamber (Neuro Probe Inc, Cabin John, Md.) was used with 8 μm pore size polycarbonate membranes (Neuro Probe Inc, Cabin John, Md.) coated with 100 μg/ml collagen type IV. 10 ng/ml VEGF or bFGF alone or with various concentrations of fibrinogen E-fragment, fibrin E-fragment, FpA or α1-24 were dissolved in DMEM+1% FCS and placed in the lower wells. The collagen-coated membrane was then placed over this and 50 μl of 25×10⁴ HuDMECs/ml (in DMEM containing 1% FCS) added to the upper chamber. The chambers were then incubated at 37° C. for 4.5 h. The chamber was then dismantled, the membrane removed and non-migrated cells scraped off the upper surface. Migrated cells on the lower surface were fixed with methanol, stained with Hema ‘Gurr’ rapid staining kit (Merck, Leics, United Kingdom) and counted using a light microscope (×160 magnification) in 3 random fields per well. Each test condition was carried out in 3-6 replicate wells and each experiment repeated 3 times.

Proliferation Assay

The MTT (3-[4,5-Dimethylthiazol-2-yl]-2,5-diphenyltetrazolium bromide) assay was used as previously described (12) to assess HuDMEC proliferation induced by VEGF or bFGF in the absence or presence of fibrinogen E-fragment, fibrin E-fragment, FpA or α1-24. HuDMEC were seeded at 3×10³ cells/100 μl in DMEM+1% FCS±10 ng/ml VEGF or bFGF in test solution into 96 well microtitre plate for 4.5 and 6 h. At these time points, a quarter volume of MTT solution (2 mg MTT/ml PBS) was added to each well and each plate was incubated for 4 h at 37° C. resulting in an insoluble purple formazan product. The medium was aspirated and the precipitates dissolved in 100 μl DMSO buffered at pH 10.5. The absorbance was then read at 540 nm on a Dynex ELISA plate reader.

Cytotoxicity Assay

HuDMECs were seeded at a density of 1-2×10⁵ cells per well in a 24 well-plate in the absence or presence of fibrinogen E-fragment, fibrin E-fragment, FpA or α1-24. After 6 h , both live (following removal by trypsinisation) and dead (floating) cells were harvested and cell viability of all cells present assessed using propidium iodide staining of 5000 cells in each of triplicate samples per treatment using a FACScan (Becton Dickinson) equipped with a blue laser excitation of 15 mW at 488 nm. The data was collected and analysed using Cell Quest software (Becton Dickinson).

In Vivo Efficacy of α1-24 Peptides

Experiments were performed on six-week-old Balb/C mice weighing 15 g, obtained from Sheffield Field Laboratories. All experiments were approved by the Home Office Project Licence Number PPL50/1414.

Tumour Cell Culture

The CT26 cell line was maintained by in vitro passage in Dulbecco's Minimal Eagles Medium containing 10% foetal calf serum, and 1% penicillin and streptomycin and maintained at 37° C. in humidified atmosphere of 5% CO₂ in air. The cell line was routinely checked to ensure freedom from mycoplasma (Mycoplasma rapid detection system, Gena-Probe Incorporated, U.S.A.).

Subcutaneous Tumour Implantation

Animals were anaesthetised with an intraperitoneal injection of diazepam (0.5 mg/ml, Dumex Ltd.) and hypnorm (fentanyl citrate 0.0315 mg/ml and fluanisone 1 mg/ml, Janssen Pharmaceutical Ltd.) in the ratio of 1:1 at a volume of 0.1 ml/200 g body weight, with supplementation as required to maintain adequate anaesthesia. Naïve Balb/c mice were immunised s.c into the right flank, following removal of the fur. Tumour cells were injected at a concentration of 3×10⁵ viable CT26 cells per animal suspended in 100 ul serum free medium. Animals were then allowed to recover. Tumour growth and animal weights were monitored daily.

Administration of Peptide α1-24

Tumour growth was measured daily and when the majority of animals in the cohort had tumour volumes of >100 mm³ but <350 mm³ animals were divided into experimental and control groups. This occurred between 14 and 18 days following implantation of the tumour cell suspension. Animals then received an intraperitoneal (ip) injection of either active drug (peptide α1-24 100 mM; 100 μl) or vehicle (phophate buffered saline, 100 μl). Daily injections continued until the tumour growth in the control animals reached the maximum burden allowed by Home Office legislation.

Assessment of Tumour Growth

Tumour volumes were assessed by calliper measurements of the perpendicular diameters and volumes estimated using the equation:

Volume=(a²×b)/2

where a is the smaller and b the larger diameter Animals were weighed on a daily basis and the general well being monitored.

Statistical Analysis.

All experiments were performed at least three times and data analysed using the Mann-Whitney U test, a non-parametric test that does not assume a Gaussian distribution in the data being analysed. P≦0.05 was taken as significant.

Endothelial cells were seen to elongate and begin to form tubules on GF-reduced Matrigel in the absence of exogenous stimuli, although it should be noted that a residual level of growth factors is present in this matrix. This assay is therefore used as a model of differentiation one of the major steps in the angiogenesis pathway. The subsequent tubule formation is measured as area covered by tubules although the number of branches yields a similar graphical pattern (data not shown). This differentiation process was significantly (p<0.001) enhanced upon the addition of either VEGF (10 ng/ml) or bFGF (10 ng/ml) to the culture medium within this assay system. Addition of the α1-24 (β-bend) to this system significantly (p<0.03) reduced tubule formation both in the absence and presence of growth factors as shown in FIG. 4.

From the results shown in FIG. 4 we would conclude that the peptide α1-24 contains the active site of the fibrinogen E-fragment. A 24 amino acid peptide is likely to have greater therapeutic utility than the more complicated, polypeptide structure of fibrinogen E-fragment, as the latter has to be made by cleavage of fibrinogen derived from blood. By contrast, the α1-24 is a smaller peptide that can be made synthetically or by recombinant techniques. It may also be possible for this to be used as part of gene therapy protocol for the treatment of cancer or other angiogenesis-dependent diseases. We then determined the in vivo efficacy of the α1-24 peptide.

In Vivo Efficacy of Peptide α1-24

### EXAMPLE 1

Experimental animals (n=6) were treated daily with ip α1-24 and control animals (n=7) with ip vehicle. The starting tumour volumes were similar in both groups of animals (experimental vs control, 235±30 vs 198±28 mm³). Tumours in the control group continued to grow at a steady rate over the 14 day period reaching a final tumour volume of 2245±371 mm³. In contrast tumours the experimental animals had a similar rate of growth until day 4, when growth was reduced. At day 7 tumours then continued to grow at a similar rate to the controls but with a reduced volume. By day 10 tumour growth again stabilised until day 14, with a final tumour volume of 1341±145 mm³ (p<0.001).

### EXAMPLE 2

Experimental animals (n=7) were treated daily with ip α1-24 and control animals (n=7) with ip vehicle. The starting tumour volumes were similar in both groups of animals (experimental vs control, 338±39 vs 300±65 mm³). Tumours in the control group continued to grow steadily over the 12 day period reaching a final tumour volume of 3072±255 mm³. In contrast, tumours in the experimental animals had a similar rate of growth to the controls until day 7, when growth stabilised until animals were killed at day 12 with a final tumour volume of 2029±504 mm³ (p<0.001).

This data, see FIG. 6, therefore demonstrates the potential of peptide α1-24 as an in vivo anti-angiogenic agent. Furthermore, peptide variants of α1-24 show similar inhibitory activity to unmodified α1-24, see FIGS. 7-9. Also, modification of α1-24 by acetylation and amidation does not affect the anti-angiogenic effect.

### References

1. Folkman J Angiogenesis in cancer, vascular, rheumatoid and other disease. Nature Medicine, 1: 27-31, 1995.

2. Leek R, Harris A L, and Lewis C E Cytokine networks in solid human tumours: regulation of angiogenesis. J. Leuk. Biol., 56: 423-35, 1994.

3. Cao Y Endogenous angiogenesis inhibitors: angiostatin, endostatin, and other proteolytic fragments. Prog Mol Subcell Biol., 20:161-76, 1998.

4. Doolittle R Fibrinogen and Fibrin. Scientific American, 245: 92-101, 1981.

5. Costantini V, Zacharski L R, Memoli V A, Kisiel W, Kudryk B J, and Rousseau S M Fibrinogen deposition without thrombin generation in primary human breast cancer tissue. Cancer Res., 51:349-53, 1991.

6. Dvorak H F, Nagy J A, Feng D, Brown L F, and Dvorak A M Vascular permeability factor/vascular endothelial growth factor and the significance of microvascular hyperpermeability in angiogenesis. Curr Top Microbiol Immunol, 237:97-132, 1999.

7. Thompson W D, Wnag J E H, Wilson S J, and Ganesalingham N Angiogenesis and fibrin degradation in human breast cancer. Angiogenesis: Molecular Biology, Clinical Aspects, 245-251, 1994.

8. Thompson W D, Smith E B, Stirk C M, Marshall F I, Stout A J, and Kocchar A Angiogenic activity of fibrin degradation products is located in fibrin fragment E. J. Pathol, 168: 47-53, 1992.

9. Malinda K M, Ponce L, Kleinman H K, Shackelton L M, and Millis A J Gp38k, a protein synthesized by vascular smooth muscle cells, stimulates directional migration of human umbilical vein endothelial cells. Exp Cell Res 250:168-73, 1999.

10. Shen J, Ham R G, Karmiol S Expression of adhesion molecules in cultured human pulmonary microvascular endothelial cells. Microvasc Res., 50:360-72, 1995.

11. Liu J, Kolath J, Anderson J, Kolar C, Lawson T A, Talmadge J, and Gmeiner W H Positive interaction between 5-FU and FdUMP[10] in the inhibition of human colorectal tumour cell proliferation. Antisense Nucleic Acid Drug Dev., 9(5):481-6, 1999.

12. Dejano E, Languino L R, Polentarutti N, Balconi G, Ryckewaert J J, Larrieu M J, Donati M B, Mantovani A, and Marguerie G Interaction between fibrinogen and cultured endothelial cells. J. Clin. Invest., 75: 11-18, 1985.

13. Bootle-Wilbraham C A, Tazzyman S, Marshall J M, Lewis C E. Fibrinogen E-fragment inhibits the migration and tubule formation of human dermal microvascular endothelial cells in vitro. Cancer Research (2000) 60: 4719-4724

14. Marsh H C, Meinwald Y C, Lee S, Martinelli R A, Scheraga H A. Mechanism of action of thrombin on fibrinogen: NMR evidence for a beta-bend at or near fibrinogen A alpha Gly(P5)-Gly(P4). Biochemistry (1985) 24: 2806-2812.

1. A polypeptide comprising an amino acid sequence selected from the group consisting of:
i) a peptide of the sequence:
A D S X E X X F L A E G G G V X X P X V V E X H
wherein X is any amino acid residue;
ii) a peptide as represented in (i) wherein amino acid residue X is selected from the following group: alanine, valine, leucine, isoleucine, proline; and
iii) a peptide represented in (i) or (ii) which has anti-angiogenic activity.

2. A polypeptide according to claim 1 wherein said polypeptide comprises an amino acid sequence selected from the following group:
A D S G E G D F L A E G G G V R G P R V V E R H
A D S G E G D F L A E G G G V R G P R V V E X H
A D S G E G D F L A E G G G V R G P X V V E R H
A D S G E G D F L A E G G G V R X P R V V E R H
A D S G E G D F L A E G G G V X G P R V V E R H
A D S G E G X F L A E G G G V R G P R V V E R H
A D S G E X D F L A E G G G V R G P R V V E R H
A D S X E G D F L A E G G G V R G P R V V E R H
A D S X E X D F L A E G G G V R X P R V V E R H
A D S G E G X F L A E G G G V R G P X V V E R H

3. A polypeptide according to claim 1 or 2 wherein X is alanine.

4. A polypeptide according to claim 1 or 2 wherein said polypeptide comprises the sequence:
A D S G E G D F L A E G G G V R G P R V V E R H.

5. A polypeptide according to any of claims 1-4 wherein the polypeptide is at least 24 amino acid residues in length.

6. A polypeptide according to any of claims 1-4 wherein the polypeptide is an active fragment of the 24 amino acid polypeptide

7. A polypeptide according to claim 5 wherein the polypeptide consists of the amino acid sequence A D S G E G D F L A E G G G V R G P R V V E R H.

8. A polypeptide according to any of claims 1-7 wherein the polypeptide is acetylated.

9. A polypeptide according to claim 8 wherein said acetylation is to the amino terminus of said polypeptide.

10. A polypeptide according to claim 9 wherein the amino terminal alanine amino acid of the α1-24 peptide is acetylated.

11. A polypeptide according to any of claims 1-7 wherein said polypeptide is amidated.

12. A polypeptide according to claim 11 wherein said amidation is to the carboxyl terminus of said polypeptide.

13. A polypeptide according to claim 12 wherein the carboxyl-terminal histidine amino acid of the α1-24 peptide is amidated.

14. A polypeptide according to any of claims 8-13 wherein said polypeptide is modified by both acetylation and amidation.

15. A polypeptide according to claim 14 wherein said acetylation is to the amino-terminal alanine amino acid of the α1-24 peptide and said amidation is to the carboxyl-terminal histidine of the α1-24 peptide.

16. A polypeptide according to any of claims 1-15 wherein said polypeptides are modified by cyclisation.

17. A nucleic acid molecule comprising DNA sequences selected from the following group:
i) the DNA sequence as represented in FIG. 6;
ii) the DNA sequence as represented in FIG. 6 which has been modified by addition, deletion or substitution of at least one nucleotide base within at least one codon to encode a peptide according to any of claims 1-6;
iii) DNA sequences which hybridise to the sequence presented in FIG. 6 which encode a peptide having anti-angiogenic activity; and
iv) DNA sequences which are degenerate as a result of the genetic code to the DNA sequences defined in (i), (ii) or (iii).

18. A nucleic acid molecule according to claim 17 wherein said nucleic acid molecule anneals under stringent hybridisation conditions.

19. A nucleic acid molecule according to claim 18 wherein stringent hybridisation conditions comprise: 4-6×SSPE; 5-10×Denhardts solution; 100 μg-1.0 mg/ml sonicated salmon/herring DNA; 0.1-1.0% sodium dodecyl sulphate; 40-60% deionised formamide; and a temperature of between 42°-65° C.

20. A pharmaceutical composition comprising at least one α1-24 peptide, or part thereof,

21. A pharmaceutical composition according to claim 20 which consists of at least one α1-24 peptide as represented by the amino acid sequences represented in FIG. 5B.

22. A pharmaceutical composition comprising at least one α1-24 polypeptide and further including at least one chemotherapeutic agent.

23. A pharmaceutical composition according to claim 22 wherein said agent is selected from the group consisting of: neocarzinostatin; cisplatin; carboplatin; cyclosphosphamide; melphalan; carmusline; methotrexate; 5-fluorouracil; cytarabine; mercaptopurine; daunorubicin; doxorubicin; epirubicin; vinblastine; vincristine; dactinomycin; mitomycin C; taxol; L-asparaginase; G-CSF; an enediyne such as chalicheamicin or esperamicin; chlorambucil; ARA-C; vindesine; bleomycin; and etoposide.

24. The use of a polypeptide comprising the α1-24 peptide, or part thereof, in the manufacture of a medicament for use in the treatment of cancer.

25. A vector which includes a nucleic acid molecule which encodes for polypeptides which comprise the α1-24 peptide, or part thereof.

26. A vector according to claim 25 wherein said vector is a prokaryotic expression vector.

27. A vector according to claim 25 wherein said vector is a eukaryotic expression vector.

28. A vector according to claim 27 wherein said vector is a gene therapy vector.

29. A vector according to claim 28 wherein said gene therapy vector is a viral based vector selected from the following viruses: adenovirus; adeno-associated virus; herpesvirus; lentivirus; vacciniavirus; baculovirus.

30. A cell which has been transformed or transfected with the nucleic acid according to any of claims 17-19 or the vector according to any of claims 25-29.

31. A method for the production of polypeptides comprising α1-24 including:
i) providing a cell according to claim 30;
ii) providing conditions conducive to the manufacture of polypeptides comprising α1-24; and
iii) purifying said polypeptides from a cell, or a cells culture environment.

32. A non-human, transgenic animal characterised in that said animal incorporates a nucleic acid molecule encoding a polypeptide comprising α1-24 into its genome.

33. A non-human, transgenic animal according to claim 32 wherein the transgene is of human origin.

34. A method to treat an animal which would benefit from inhibition of angiogenesis comprising:
i) administering an effective amount of an agent comprising α1-24 to an animal to be treated;
ii). monitoring the effects of said agent on the inhibition of angiogenesis.

35. A method to treat an animal which would benefit from inhibition of angiogenesis comprising:
i) administering an effective amount of a pharmaceutical composition according to claim 20 or 21 to an animal to be treated;
ii). monitoring the effects of said composition on the inhibition of angiogenesis.

36. A method according to claim 34 or 35 wherein polypeptides comprising α1-24 peptides are conjugated, associated or crosslinked to a chemotherapeutic agent.

37. A method to treat an animal which would benefit from inhibition of angiogenesis comprising:
i) administering an effective amount of the nucleic acid according to any of claims 17-19 or the vector according to claim 28 or 29; and
ii) monitoring the effects of transfection of the nucleic acid in (i) on the inhibition of angiogenesis.

38. A method according to any of claims 34-37 wherein said treatment is the inhibition of tumour angiogenesis.

39. A method according to any of claims 34-38 wherein said animal is human.

40. An imaging agent which comprises an α1-24 polypeptide conjugated, coupled, associated or crosslinked to a detectable label.
